(19) United States
(12) Patent Application Publication
(10) Pub. No.: US 2024/0065868 A1
(43) Pub. Date: Feb. 29, 2024

(54) DEVICES, SYSTEMS, AND METHODS FOR IMPLANTABLE MEDICAL DEVICES

(71) Applicant: BOSTON SCIENTIFIC SCIMED, INC., (US)

(72) Inventors: Ryan Desmond Lynch, Roscommon (IE); Fionn Stapleton, Galway (IE); John O'Driscoll, Galway (IE); Aran Murphy, Wexford (IE); Darren Gerard Curran, Galway (IE); Emma Horgan, Galway (IE)

(73) Assignee: BOSTON SCIENTIFIC SCIMED, INC., MAPLE GROVE, MN (US)

(21) Appl. No.: 18/240,112

(22) Filed: Aug. 30, 2023

Related U.S. Application Data
(60) Provisional application No. 63/402,698, filed on Aug. 31, 2022.

(51) Int. Cl. A61F 5/00; A61B 17/11; A61F 2/04

(52) U.S. Cl. CPC A61F 5/0079; A61B 17/1114; A61F 2/04; A61F 2002/045; A61F 2220/0008

(57) ABSTRACT

An implantable medical device system having at least two implantable medical devices coupled together with a connector while implanted at a common anatomical region. The connector allows the implantable medical devices to anchor one another in place at respective deployment sites. Moreover, the connector may function as a safety device by causing one implantable medical device to migrate upon migration of another implantable medical device. For instance, if migration of an implantable medical device causes a structural change in the common anatomical region, such change may be generally reversible upon migration of the other implantable medical device as a result of the implantable medical devices being coupled via the connector.

### CROSS-REFERENCE TO RELATED APPLICATIONS

This application claims the benefit of priority under 35 U.S.C. § 119 to U.S. Provisional Application No. 63/402,698, filed Aug. 31, 2022, the entire disclosure of which is hereby incorporated by reference herein for all purposes.

### FIELD

This disclosure relates generally to the field of implantable medical devices. More particularly, this disclosure relates to devices, systems, and methods addressing forces which may cause migration of implantable devices. Even more particularly, the present disclosure relates to devices, systems, and methods preventing migration of implantable medical devices and/or providing safety mechanisms upon migration of implantable medical devices.

### BACKGROUND

Various implantable medical devices are known for extending across anatomical structures for various purposes. For instance, various stents may be used to hold tissue in apposition, and/or to affect (e.g., establish or occlude) connections (e.g., fluid communication) between anatomical structures such as organs, cavities, lumens, passages, etc. In some instances, it is desirable to create a semi-permanent or permanent anastomosis with an implantable medical device to allow fluid flow or drainage from one anatomical structure to another anatomical structure. Additionally or alternatively, in some instances it is desirable to occlude or partially occlude a body passage with an implantable medical device. In various procedures, lumen-apposing stents may be used to form an anastomosis between anatomical structure and/or to affect flow of materials through a body passage. For instance, gastrointestinal (GI) procedures such as bypass surgery use a lumen-apposing stent to create a gastrojejunostomy between the stomach and the jejunum to facilitate flow of food particulate, liquid, chyme, etc. (generally, “gastric materials”), from the stomach to the lower GI tract. Additionally, such procedure generally involves bypassing the pylorus and the duodenum (e.g., approximately the first 1.5 m of the small intestine, where most food, fats, and nutrients are digested) by deploying another device across the pylorus to occlude flow therethrough. As such, gastric materials are inhibited or prevented from passing into (and being digested in) the duodenum, and are redirected to the anastomosis between the stomach and the jejunum to bypass the duodenum. Such endoscopic by-pass procedure is considered to be less invasive than prior Roux-en-Y surgical bypass procedures, and may be reversible. The first implantable medical device may be helpful in maintaining patency of the anastomosis. Accordingly, it may be desirable for at least the first stent to remain securely in place until removal is desired or medically indicated. Further, it may be desirable to maintain the second implantable medical device in place to maintain the redirected flow of gastric materials to the jejunum rather than to the duodenum. Moreover, if the first implantable medical device is displaced and the anastomosis closes, it may be important to provide an alternative pathway for gastric materials to flow to the intestines, such as through the pylorus. Accordingly, there is an ongoing need for improvements to maintaining implantable medical devices, systems, and methods in place and/or addressing any displacement thereof.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for forming a gastric bypass in a patient includes a first implantable medical device configured to form an anastomosis between the stomach and a portion of the jejunum of the patient; a second implantable medical device configured to occlude the pylorus of the patient; and a connector coupling the first implantable medical device and the second implantable medical device.

In some aspects, the connector has a configuration distinct from the configurations of the first implantable medical device and the second implantable medical device.

In some embodiments, the connector is elongated and has a length capable of extending from the first implantable medical device when implanted to create an anastomosis between the stomach and the jejunum and the second implantable medical device when implanted across the pylorus.

In some embodiments, the first implantable medical device is a stent configured to maintain patency of the anastomosis, the stent comprising a saddle region configured to extend through the stomach wall and a portion of a wall of the jejunum, a proximal retention member extending radially outwardly from a proximal end of the saddle region and configured to be seated within the stomach to inhibit distal migration of the first implantable medical device away from the stomach, and a distal retention member extending radially outwardly from a distal end of the saddle region and configured to be seated within the jejunum to inhibit proximal migration of the first implantable medical device into the stomach. In some embodiments, the second implantable medical device has a saddle region configured to occlude flow of material therethrough, a proximal retention member extending radially outwardly from a proximal end of the saddle region and configured to be seated within the stomach to inhibit distal migration of the second implantable medical device away from the stomach, and a distal retention member extending radially outwardly from a distal end of the saddle region and configured to be seated within the duodenum of the patient to inhibit proximal migration of the second implantable medical device into the stomach.

In some embodiments, the connector is an elongated element coupling the first implantable medical device and the second implantable medical device. In some embodiments, the connector is coupled to a proximal retention member of at least one of the first implantable medical device and the second implantable medical device.

In some embodiments, the connector includes an elongated portion having a first end and a second end, and a connector bar coupled to one of the first end or the second end of the elongated portion and movable between a first position extending along the elongated portion and a second position transverse to the elongated portion, the connector bar being extendable through a portion of a wall of one of the first implantable medical device or the second implantable medical device when in the first position, and movable to the second position to hold the one of the first end or the second end of the elongated portion coupled to the portion of a wall of one of the first implantable medical device or the second implantable medical device.

In some embodiments, one of the first implantable medical device or the second implantable medical device has at least one double-walled retention member comprising an inner wall adjacent the saddle region and an outer wall spaced away from the inner wall and defining a space between the inner wall and the outer wall; and the connector bar is extendable into the space between the inner wall and the outer wall of the double-walled retention member and movable to the second position to be retained in the space.

In some embodiments, the connector is formed separately from the first implantable medical device and the second implantable medical device.

In some aspects, the first implantable medical device anchors, via the connector, the second implantable medical device against migrating distally from the stomach; and the second implantable medical device anchors, via the connector, the first implantable medical device against migrating distally from the stomach.

In some aspects, the first implantable medical device displaces the second implantable medical device upon migrating from the anastomosis to open the pylorus for passage of gastric materials therethrough.

In accordance with various principles of the present disclosure, a system is provided for maintaining a pair of implantable devices in place with respect to respective deployment sites for the pair of implantable medical devices. The system includes a first implantable medical device; a second implantable medical device; and a connector coupling the first implantable medical device and the second implantable medical device. In some aspects, the deployment sites are at anatomical environments tending to drive the first implantable medical device and the second implantable medical device apart from each other; and each of the implantable medical devices holds the other of the implantable medical devices in place via the connector.

In some embodiments, the connector has a configuration distinct from the configurations of the first implantable medical device and the second implantable medical device.

In some embodiments, the connector is elongated and has a length sufficient to extend between the first implantable medical device and the second implantable medical device without exerting force on either of the first implantable medical device and the second implantable medical upon coupling the first implantable medical device and the second implantable medical.

In some embodiments, the connector is formed separately from the first implantable medical device and the second implantable medical device.

In some embodiments, the connector includes an elongated portion having a first end and a second end, and a connector bar coupled to one of the first end or the second end of the elongated portion and movable between a first position extending along the elongated portion and a second position transverse to the elongated portion, the connector bar being extendable through a portion of a wall of one of the first implantable medical device or the second implantable medical device when in the first position, and movable to the second position to hold the one of the first end or the second end of the elongated portion coupled to the portion of a wall of one of the first implantable medical device or the second implantable medical device.

In accordance with various principles of the present disclosure, a method of forming a gastric bypass includes implanting a first implantable device across an apposed portion of a stomach wall and portion of a jejunum to form an anastomosis between the stomach and the jejunum; implanting a second implantable medical device in a pylorus to at least partially occlude the pylorus; and coupling the first implantable medical device and the second implantable medical device with an elongated connector.

In some aspects, the method further includes implanting the first implantable medical device on a first day, and implanting the second implantable medical device on a second day different from the first day.

In some aspects, coupling the first implantable medical device and the second implantable medical device with an elongated connector is performed after implanting the first implantable medical device and the second implantable medical device.

In some embodiments, the method further includes anchoring the second implantable medical device against migrating distally from the stomach via the connector and the first implantable medical device, and anchoring the first implantable medical device against migrating distally from the stomach via the connector and the first implantable medical device.

In some embodiments, the method further includes coupling the connector between the first implantable medical device and the second implantable medical device so that the first implantable medical device displaces the second implantable medical device upon migrating from the anastomosis to open the pylorus for passage of gastric materials therethrough.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an implantable medical device system formed in accordance with various aspects of the present disclosure and positioned in a schematic representation of a gastrointestinal environment.

FIG. 2 illustrates a perspective view of an example of an embodiment of a connector of an implantable medical device system formed in accordance with various principles of the present disclosure.

FIG. 3 illustrates a perspective view of a portion of a connector such as illustrated in FIG. 2 engaged with an example of an embodiment of an implantable medical device which may be used in a system such as illustrated in FIG. 1.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Various medical procedures involve deployment of two or more implantable medical devices in an anatomical region, such as a common anatomical region. It will be appreciated that reference to a common anatomical region may encompass a common organ, cavity, body passage, etc., as well as adjacent organs, cavities, body passages, etc. The implantable medical devices may be subject to various biological forces, such as internal (e.g., peristalsis, blood flow, or other fluid flows, etc.) or external (e.g., external body movement affecting internal organs) movements. Generally, it is desirable to maintain the position of the implantable medical devices at the site at which it has been deployed. It will be appreciated that reference may be made herein to terms such as deployed, implanted, anchored, etc., including various grammatical forms thereof, interchangeably and without intent to limit. For instance, reference may be made interchangeably to a deployment site, an implant site, etc., without intent to limit. Two or more implantable medical devices implanted in an anatomical region may be subject to the same forces, e.g., in the same general direction with respect to the anatomical region. For instance, the implantable medical devices may be subject to similar forces driving the implantable medical devices out of the anatomical region in which the devices have been deployed. Such movement may also be movement of the devices away from one another.

In accordance with various principles of the present disclosure, two or more implantable medical devices are implanted in a common anatomical region, spaced apart from one another yet also coupled together. More particularly, in some embodiments, the implantable medical devices are separately formed and/or independently implanted, with the connection therebetween being achieved by a structure, such as a connector, distinct or differentiable from the implantable medical devices. For instance, one or more of the implantable medical devices may be a treatment device having a structure interacting with the anatomical region, whereas the connector may have a simple role of connecting the implantable medical devices without otherwise affecting the anatomical region. More particularly, the implantable medical devices may be medical devices having structures facilitating a particular treatment course or therapy or procedure (such terms and other terms being used interchangeably herein without intent to limit), whereas the connector may be a simple elongated element configured to connect the implantable medical devices together. In some embodiments, the connector may be an elongated element structurally different from the implantable medical devices. The connector may be a tether, wire, suture, cord, band, etc., without limitation other than the ability to maintain connection between the implantable medical devices.

In some embodiments, a separately formed connector (e.g., not an integrally formed section or portion of any of the implantable medical devices) may be used to couple implantable medical devices together in accordance with various principles of the present disclosure. For example, a separately formed connector may be engaged with the implantable medical devices to couple the implantable medical devices together. However, given the different structures and functions of the implantable medical devices and the connector, in some embodiments the connector may be formed as an extension of at least one of the implantable medical devices (e.g., integrally formed therewith), but having a unique configuration (e.g., elongated) differentiated and/or distinct from the general shape and configuration of the implantable medical devices. It will be appreciated that in accordance with various principles of the present disclosure, the implantable medical devices may be connected after at least one of the implantable medical devices is implanted, or after all implantable medical devices are implanted, or may be coupled with one or more of the implantable medical devices before deployment.

In some instance, forces from a common anatomical region in which implantable medical devices are implanted may tend to dislodge one or both of the implantable medical devices. An implantable medical device system including two or more implantable medical devices and a connector provides a migration prevention and/or safety system for implantable medical devices. For the sake of simplicity, and without intent to limit, reference will be made hereinafter to two implantable medical devices. An implantable medical device system formed in accordance with various principles of the present disclosure includes implantable medical devices implanted in a common anatomical region and connected to one another with a connector so that one of the implantable medical devices may anchor the other of the implantable medical devices against forces tending to cause the other of the implantable medical devices to migrate from its deployment site. In such use, the one of the implantable medical devices may be implanted at a location and/or formed in a manner which facilitates a stronger hold with respect to the implant site than may be achieved by the other of the implantable medical devices. Additionally or alternatively, the other of the implantable medical devices may be implanted at a location which has a greater tendency to allow migration thereof. Of course, it will be appreciated, that the tendency to migrate may be simply situational, such as based on the nature of a particular force impacting one of the implantable medical devices more than the others.

Additionally or alternatively, migration of one the implantable medical devices may create a need for displacement of the other of the implantable medical devices so that migration of the one of the implantable medical device causes the other implantable medical device to migrate as a manner of a safety release. For instance, the one of the implantable medical devices may migrate in one direction with respect to the anatomical region and pull the other of the implantable medical devices along with it. More particularly, the implantable medical devices may be implanted in a common anatomical region, one of the implantable medical devices may migrate out of the anatomical region, initially pulling the other of the implantable medical devices into the anatomical region (thus moving in an opposite direction with respect to the anatomical region than the one of the implantable medical devices migrates), and then out of the anatomical region with the one of the implantable medical devices. As a result, migration of a first of implantable medical device of an implantable medical device system formed in accordance with various principles of the present disclosure may cause ultimate migration and removal of the entire system. Such configuration may be useful if migration of one of the implantable medical devices of a system formed in accordance with various principles of the present disclosure migrates without others of the implantable medical devices migrating as well. For instance, in some uses, migration of one of the implantable medical devices may cause a structural change to or along the common anatomical region at which the implantable medical device system is implanted. Such change may be reversible by the implantable medical device causing the structural change (upon migration) to also cause the other implantable medical device of the system to migrate, such as via the connector between the implantable medical devices.

In some embodiments, one of the implantable medical devices of an implantable medical device system formed in accordance with various principles of the present disclosure may be structured to maintain patency of an outlet passage from a common anatomical region at which the implantable medical device system is implanted. Another of the implantable medical devices of an implantable medical device system formed in accordance with various principles of the present disclosure may be structured to occlude an alternate passage from the common anatomical region. In such configuration of an implantable medical device system, it may be desirable upon migration of the implantable medical device from the anastomosis to cause the occluding implantable medical device to migrate as well. Specifically, if the implantable medical device maintaining patency of the only outlet passage from the common anatomical region migrates, and such passage closes, then it may be desirable for the occluding implantable medical device to be removed from occluding the other outlet passage from the common anatomical region. In such embodiment, the connector between such implantable medical devices allows migration of the implantable medical device from the anastomosis to cause migration of the implantable medical device from occluding the other outlet. As such, the implantable medical device system may be considered to form a safety feature/system.

Various embodiments of implantable medical devices and system, and associated methods of use thereof, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and sub combinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

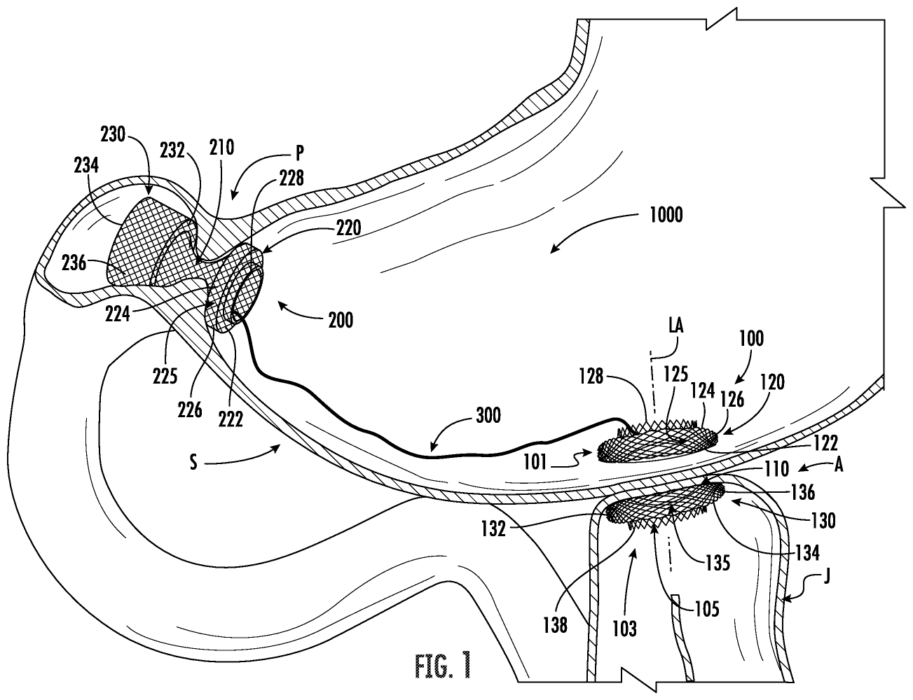

Turning now to the drawings, an implantable medical device system 1000 formed in accordance with various principles of the present disclosure is illustrated in FIG. 1 implanted in a schematic example of an embodiment of a common anatomical region. Specifically, the implantable medical device system 1000 is illustrated in a gastric system, with a first implantable medical device 100 thereof implanted to create an anastomosis A between a stomach S and a jejunum J, a second implantable medical device 200 thereof implanted across a pylorus P (between the stomach S and a duodenum D), and a connector 300 coupling the first implantable medical device 100 and the second implantable medical device 200 together. In some aspects, the connector 300 has a length sufficient to extend between the first implantable medical device 100 and the second implantable medical device 200 without itself exerting force on either as a result of simply connecting the implantable medical devices 100, 200 and/or under normal use of the implantable medical device system 1000. In other words, the connector 300 may have a length such that the connector 300 exerts forces on one of the implantable medical devices 100, 200 via movement of the other of the implantable medical devices 100, 200 and not as a result of being shorter than the distance between the implantable medical devices 100, 200. In some embodiments, the connector 300 is at least as long as the distance between the implantable medical devices 100, 200 when the implantable medical devices 100, 200 are implanted (such as illustrated in FIG. 1). In some embodiments, the length of the connector 300 is such that the connector 300 does not exert force on either of the implantable medical devices 100, 200 when the implantable medical device system 1000 is deployed and no migration forces are being exerted on the implantable medical devices 100, 200. In such embodiment, once a migration force is exerted on one of the implantable medical devices 100, 200, such as to affect the position thereof, the connector 300 exerts a force to resist the migration force to maintain the deployed position of the implantable medical device 100, 200.

More particularly, each of the first implantable medical device 100 and the second implantable medical device 200 has a proximal end 101, 201 deployed within the common anatomical region (in this case, within the stomach S), and a distal end 103, 203 extending away from the common anatomical region. In the example of an embodiment illustrated in FIG. 1, the distal ends 103, 203 are positioned in adjacent anatomical regions (in this case, the jejunum J and the duodenum D, respectively). As may be appreciated, the implantable medical devices 100, 200 each include a saddle region 110, 210 extending the implantable medical devices 100, 200 from one anatomical region to another. For instance, in the illustrated example of an embodiment of a first implantable medical device 100, the saddle region 110 extends across the apposed walls of the stomach S and the jejunum J. Furthermore, in the illustrated example of an embodiment of a second implantable medical device 200, the saddle region 210 extends across the pylorus P between the stomach S and the duodenum D.

In some embodiments, such as the embodiments illustrated herewith, each of the implantable medical devices 100, 200 includes a proximal retention member 120, 220, respectively, configured to retain the implantable medical devices 100, 200 in place with respect to the common anatomical region at which the implantable medical devices 100, 200 are deployed. More particularly, the proximal retention members 120, 220 may be sized, shaped, configured, and/or dimensioned to resist forces against the implantable medical devices 100, 200 which may cause the implantable medical devices 100, 200 to migrate from their respective deployment sites. Even more particularly, the proximal retention members 120, 220 of the implantable medical devices 100, 200 may be configured to resist movement of the implantable medical devices 100, 200 in a direction away from the anatomical structure (in the illustrated example, the stomach S) in which the proximal retention members 120, 220 are positioned. In the example of an embodiment illustrated in FIG. 1, one or both of the implantable medical devices 100, 200 may have a proximal retention member 120, 220 which extends radially outwardly from a respective saddle region 110, 210 thereof, such as to seat against the proximal tissue wall surrounding the passage through which the respective saddle regions 110, 210 of the implantable medical devices 100, 200 extend. Typically, the proximal retention members 120, 220 resist migration of the respective implantable medical devices 100, 200, respectively, in a distal direction.

Optionally, each of the implantable medical devices 100, 200 includes a distal retention member 130, 230, respectively, configured to retain the implantable medical devices 100, 200 in place with respect to the site at which the implantable medical devices 100, 200 are deployed. More particularly, the distal retention members 130, 230 may be sized, shaped, configured, and/or dimensioned to resist forces against the implantable medical devices 100, 200 which may cause the implantable medical devices 100, 200 to migrate from their respective deployment sites. Even more particularly, the distal retention members 130, 230 of the implantable medical devices 100, 200 may be configured to resist movement of the implantable medical devices 100, 200 in a direction away from the anatomical structure (in the illustrated example, the jejunum J and the duodenum D, respectively) in which the distal retention members 130, 230 are positioned. In the example of an embodiment illustrated in FIG. 1, one or both of the implantable medical devices 100, 200 may have a distal retention member 130, 230 which extends radially outwardly from a respective saddle region 110, 210 thereof, such as to seat against the distal tissue wall surrounding the passage through which the respective saddle regions 110, 210 of the implantable medical devices 100, 200 extend. Typically, the distal retention members 130, 230 resist migration of the respective implantable medical devices 100, 200, respectively, in a proximal direction.

In view of the above, it will be appreciated that in the example of an embodiment illustrated in FIG. 1, each of the implantable medical devices 100, 200 is configured to be implanted with respect to an anatomical region and to resist migration with respect to such anatomical region. In accordance with various principles of the present disclosure, a connector 300 is coupled to the implantable medical devices 100, 200 so that the implantable medical devices 100, 200 and the connector 300 are, together, an implantable medical device system 1000 resisting migration from the deployment site thereof. In the example of an embodiment illustrated in FIG. 1, the deployment site of the implantable medical device system 1000 is in a common anatomical structure such as the stomach S. However, the deployment site may be a common anatomical region which includes adjacent anatomical structures as well, with the implantable medical device system 1000 extending through one or more anatomical structure. For instance, the first implantable medical device 100 may be deployed in a first anatomical structure, and the second implantable medical device 200 may be deployed in a second anatomical structure with the connector 300 extending therebetween (in either or both of the first and second anatomical structure, or through another anatomical structure, such as a lumen or passage, between the first and second anatomical structures). The implantable medical device system 1000 utilizes the components thereof to resist migration by utilizing the anti-migration feature of one of the implantable medical devices 100, 200 to anchor the other of the implantable medical devices 100, 200. For instance, if one of the implantable medical devices 100, 200 has a greater tendency or likelihood to migrate (e.g., because of a difference in its structure, and/or being subjected to greater migration-inducing forces, and/or a difference in the nature of its implant site compared with the implant site of the other of the implantable medical devices 100, 200, etc.) than the other of the implantable medical devices 100, 200, then the one of the implantable medical devices 100, 200 may act as an anchor for the other of the implantable medical devices 100, 200 via the connector 300. More particularly, if one of the implantable medical devices 100, 200 is inclined to migrate from the common anatomical region at which the implantable medical devices 100, 200 are implanted, such one of the implantable medical devices 100, 200 may also be inclined to migrate away from the other of the implantable medical devices 100, 200. When the other of the implantable medical devices 100, 200 is securely implanted at its particular deployment site, the other of the implantable medical devices 100, 200 may hold the one of the implantable medical devices 100, 200 in place, via the connector 300, against migration. Additionally or alternatively, if the other of the implantable medical devices 100, 200 is also subjected to a force which drives the other of the implantable medical devices 100, 200 away from the common anatomical region at which the implantable medical device system 1000 has been implanted, and thus away from the one of the implantable medical devices 100, 200, then both of the implantable medical devices 100, 200 may exert anchoring forces on each other or otherwise resist migration of the implantable medical device 100, 200 to which it is coupled via the connector 300. As such, use of a connector 300 in accordance with various principles of the present disclosure with two or more implantable medical devices forms an anti-migratory implantable medical device system 1000. The connector 300 may be particularly useful in preventing migration of one of the implantable medical devices 100, 200 in a direction away from the other of the implantable medical devices 100, 200.

It will be appreciated that the connector 300 of the implantable medical device system 1000 may be coupled to any of a variety of sections or portions of an implantable medical device 100, 200. For instance, the connector 300 may be coupled to one of the retention members 120, 130, 220, 230, and/or to one of the saddle regions 110, 210 of the implantable medical devices 100, 200. Moreover, the free ends of the connector 300 or another portion of the connector 300 may engage the implantable medical devices 100, 200 to couple the connector 300 with the implantable medical devices 100, 200 in any of a variety of manners. For instance, the connector 300 may be tied, looped, welded, bonded, sewn, knotted, interwoven, or otherwise coupled to the implantable medical devices 100, 200. It will be appreciated that terms such as coupled, connected, engaged, interengaged, etc., and other grammatical forms thereof may be used interchangeably herein without intent to limit. Additionally, it will be appreciated that the connector 300 may be coupled to the implantable medical devices 100, 200 in a manner selected to counteract particular migratory forces anticipated to impact the implantable medical device system 1000.

The connector 300 may be any generally elongated structure capable of coupling the implantable medical devices 100, 200 together, such as spaced apart from each other, and maintaining the implantable medical devices 100, 200 coupled together. The connector 300 may be formed of a wire; a suture; a filament, a thread; an elastic or semi-elastic or non-elastic polymeric tether or band; etc. Preferably, the connector 300 is formed of a biocompatible material and is capable of withstanding various anatomical forces and/or environments (including chemical forces, such as from gastric juices). Examples of materials which may be used to form the connector 300 include, without limitation, silicone, polyethylene terephthalate (PET), various nonabsorbable suture materials (smooth or rough, elastic or nonelastic, etc.), etc.

In some embodiments, the ends of the connector 300 are enlarged or otherwise adjustable with respect to a portion of an implantable medical device 100, 200 to be engaged therewith. Such enlarged or adjustable end may facilitate coupling of the connector 300 to an implantable medical device 100, 200 as well as resist decoupling therefrom. For instance, one or both ends of the connector 300 may be engageable with a portion of one or both of the implantable medical devices 100, 200 in a manner which couples the connector 300 with the implantable medical device 100, 200. An end of the connector 300 may be formed, shaped, configured, etc., in a manner which allows coupling of the connector 300 with the implantable medical device 100, 200 so that the connector 300 and the implantable medical device 100, 200 do not inadvertently decouple. For instance, an end of the connector 300 may be passed through a wall of an implantable medical device 100, 200 and enlarged or otherwise formed, shaped, configured, etc., to resist passing back out of the wall of the implantable medical device 100, 200. In some embodiments, the enlarged end is in the form of a knot, crimp, weld, etc.

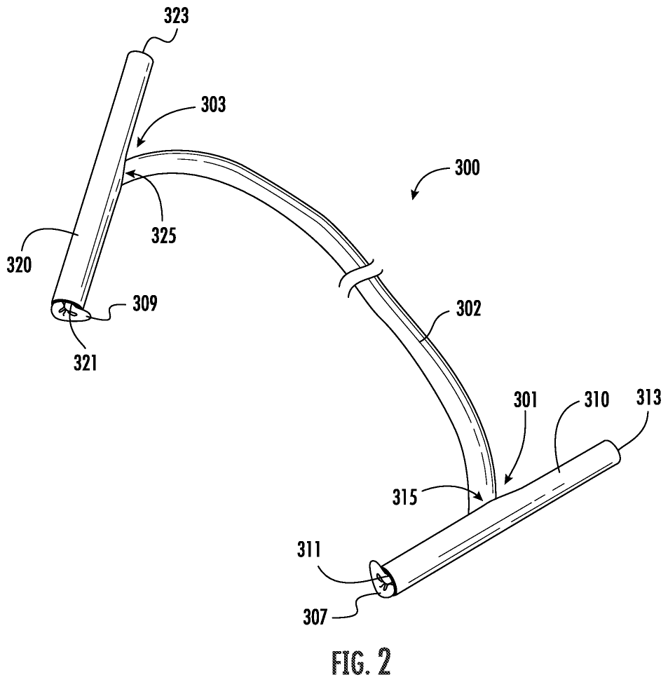

In some embodiments, an end of a connector 300 is selectively movable into a configuration which maintains such end in place with respect to an implantable medical device 100, 200. For instance, in an example of an embodiment of a connector 300 illustrated in FIG. 2, one or both ends 301, 303 of a connector 300 are coupled with a catch bar 310, 320, respectively. The catch bar 310, 320 may be movable (e.g., may toggle) between a position generally aligned with the elongated portion 302 of the connector 300 between the ends 301, 303 thereof, and a catch position generally transverse to (e.g., perpendicular to) the elongated portion 302 of the connector 300 (such as illustrated in FIG. 2). As such, either catch bar 310, 320 may pass through a wall of a portion of an implantable medical device 100, 200, such as when the catch bar 310, 320 is generally aligned with the elongated portion 302 of the connector 300, and then be moved into a catch position to resist movement out of engagement with the implantable medical device 100, 200. It will be appreciated that the ends 301, 303 of the connector 300 may be coupled to the catch bar 310, 320 along free ends of the connector 300 (the terminal ends of the elongated portion 302 of the connector 300), such as against (e.g., in abutment with) a surface of the catch bar 310, 320. Alternatively, the ends 301, 303 of the connector 300 may be passed through an aperture in a wall of a generally tubular catch bar 310, 320 (e.g., along a respective middle section 315, 325 of the catch bar 310, 320 between either of the ends 311, 313, 321, 323 thereof) and secured therein. For example, a free end 307, 309 of the connector 300 may extend out of an end 311, 321 of the catch bar 310, 320 and be expanded to resist withdrawal therefrom, such as illustrated in FIG. 2. Additionally or alternatively, an end 301, 303 of the connector 300 may be coupled within the lumen of a generally tubular catch bar 310, 320 such as with adhesive, a weld, a friction or interference fit, etc.

One or both of the implantable medical devices 100, 200 of an implantable medical device system 1000 formed in accordance with various principles of the present disclosure may be formed in a variety of manners. In some embodiments, the implantable medical devices 100, 200 are scaffold or stent structures which are expandable from a collapsed or contracted delivery configuration (e.g., facilitating intralumenal delivery to a deployment site through body passages without requiring open surgery for delivery of the device) to an expanded deployed configuration (such as illustrated in FIG. 1). In some embodiments, one or both of the implantable medical device 100, 200 are formed from one or more members/elements (such terms being used interchangeably herein without intent to limit) combined to form a rigid and/or semi-rigid structure. In some embodiments, the walls of the implantable medical device have gaps, apertures, openings, interstices, etc., therethrough. Application of a coating material to, over, on, etc. (such terms being used interchangeably herein without intent to limit), at least a portion of the device wall may fill in such gaps, apertures, openings, interstices, etc., to inhibit or prevent flow or leakage of materials therethrough and/or to inhibit tissue ingrowth therein. The members forming the device (e.g., the wall of the device) may be formed of one or more struts, wires, strands, filaments, etc., such terms being used interchangeably herein without intent to limit. For the sake of convenience, and without intent to limit, reference is made to filaments which may be braided, interengaged, intertwined, interwoven, knitted, knotted, looped (e.g., bobbinet-style), weaved, woven, wrapped, or the like to form a scaffold configuration which may be expandable and/or contractable. The filaments forming an implantable medical device of an implantable medical device system 1000 of the present disclosure may be formed from a variety of non-limiting preferably biocompatible materials, such as, without limitation, a metal, metal alloy, polymer, metal-polymer composite, ceramics, and combinations or subcombinations thereof. For instance, the filaments forming the implantable medical device may be formed from a variety of non-limiting preferably biocompatible polymers, such as, without limitation, polypropylene, polyester, polysulfone, nylon, silicones, polyurethane, polystyrene, polyethylene (PE) (including high-density and low-density PE's), polyethylene terephthalate (PET), polyethylene naphthalate (PEN), polybutylene terephthalate (PBT), polytrimethylene terephthalate, polyether block amides (PEBA), polyetheretherketone (PEEK), polyetherimide (PEI), poly(methyl methacrylate) (PMMA), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly paraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, subcombinations, and copolymers thereof. Additionally or alternatively, the members forming the implantable medical device may be formed from a variety of non-limiting preferably biocompatible metals, such as, without limitation, stainless steel, a nickel-titanium alloy such as Nitinol, a nickel-tungsten or tungsten alloy, a cobalt-chromium alloy, a cobalt-chromium-nickel based alloy such as Elgiloy®, a nickel-copper alloy, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and subcombinations and other alloys thereof. Additionally or alternatively, the members forming an implantable medical device of an implantable medical device system 1000 of the present disclosure may be formed from a variety of non-limiting preferably biocompatible natural materials, such as, without limitation, cat or bovine intestine, or the like; a natural fiber, such as silk or cotton, or the like, and combinations and subcombinations thereof. It will be appreciated that the members forming an implantable medical device of an implantable medical device system 1000 of the present disclosure may be formed from mixtures, composites, combinations, subcombinations, copolymers, or co-constructions of any of the above. Alternatively or additionally, the members forming the implantable medical device may be formed by cutting (e.g., by laser-cutting) a tubular structure (e.g., an, optionally monolithic, cylindrical tubular member) into an expandable configuration, the cuts forming members such as strut members. An implantable medical device of an implantable medical device system 1000 of the present disclosure may be a self-expanding device such as known or heretofore known to those of ordinary skill in the art. For instance, the implantable medical device may be formed of shape-memory or heat-formable material (e.g., Nitinol or Elgiloy® or shape memory polymers) so that the implantable medical device returns to a pre-shaped expanded configuration from a collapsed configuration upon advancement from a delivery sheath (any acceptable tubular elongated member such as known to those of ordinary skill in the art for delivery of medical devices) and/or withdrawal of a delivery sheath which maintains the implantable medical device in a delivery configuration therein.

In view of the above, at least a portion of an implantable medical device 100, 200 of an implantable medical device system 1000 formed in accordance with various principles of the present disclosure may have interstices or openings through which an end of a connector 300 may be inserted to be coupled with the implantable medical device 100, 200. As such, an end of a connector 300 of an implantable medical device system 1000 may be passed through a portion of a wall of an implantable medical device 100, 200 to be coupled therewith. For instance, the end of the connector 300 may be passed through a wall of and subsequently bonded or otherwise attached to a wall of an implantable medical device 100, 200, such as described above, or the end of the connector 300 may be enlarged and thereby inhibited or prevented from being withdrawn from the implantable medical device 100, 200. More particularly, an end of a connector 300 such as illustrated in FIG. 2 may be inserted through a portion of an implantable medical devices 100, 200 (e.g., through an opening in a wall thereof) with the catch bar 310, 320 generally aligned with the elongated portion 302 of the connector 300. Once the catch bar 310, 320 has passed through a portion (e.g., a portion of a wall) of an implantable medical device 100, 200, the catch bar 310, 320 is transitioned to a position generally transverse to the elongated portion 302 of the connector 300 to generally lock the end of the connector 300 in place with respect to the implantable medical device 100, 200.

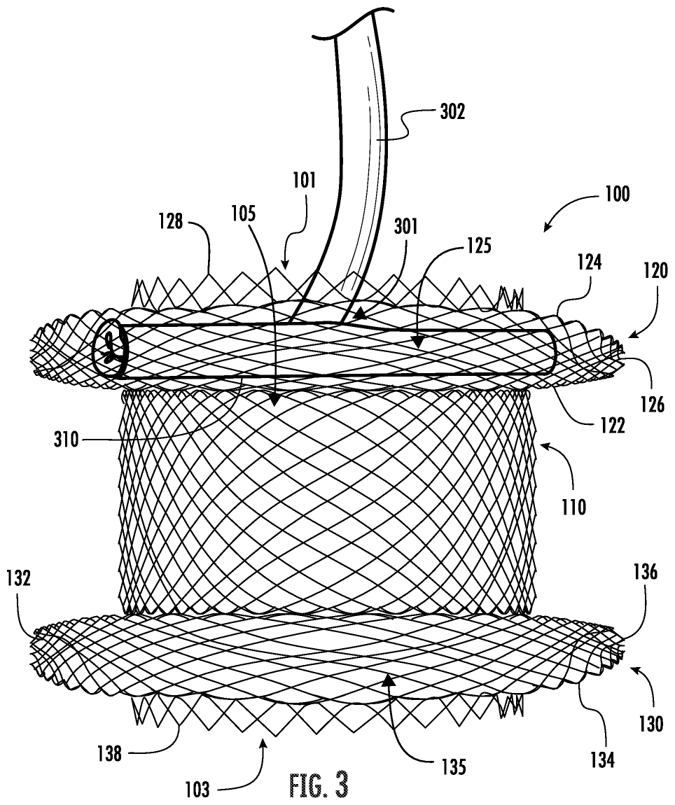

In some embodiments, such as illustrated in FIG. 3, an implantable medical device 100 has at least one retention member 120, 130 formed as a double-wall retention member 120, 130. More particularly, in the example of an embodiment illustrated in FIG. 3, the proximal retention member 120 includes an inner wall 122 (closer to the saddle region 110) and an outer wall 124 generally spaced apart from the inner wall 122. A peripheral wall 126 may extend between the inner wall 122 and the outer wall 124, such as to maintain a space 125 between the walls 122, 124 of the proximal retention member 120. The distal retention member 130 may similarly include an inner wall 132 (closer to the saddle region 110) and an outer wall 134 (generally spaced apart from the inner wall 132), and an optional peripheral wall 136 extending between the inner wall 132 and the outer wall 134, with a space 135 between the walls 132, 134. As may be appreciated with reference to FIG. 3, a catch bar 310 coupled to an end 301 of the elongated portion 302 of the connector 300 may be extended into the space 125 within the proximal retention member 120 and oriented to resist being removed from the space 125. The catch bar 310 may be passed through the outer wall 124 in a position generally along the elongated portion 302 of the connector 300 and then transitioned to a position transverse to the elongate portion 302 such as illustrated. The catch bar 310 may similarly be positioned and retained within the space 135 between the walls 132, 134 of the distal retention member 130. The other catch bar 320 of the connector 300 (illustrated in FIG. 2) may be positioned and retained with respect to the second implantable medical device 200 in a similar manner. For instance, the proximal retention member 220 of the second implantable medical device 200 may have an inner wall 224 and an outer wall 226 spaced apart from each other (optionally by a peripheral wall or lip 228) to define a space 225 in which the other catch bar 320 of the connector 300 may be inserted and retained to connect the implantable medical devices 100, 200.

It will be appreciated that the present disclosure encompasses other configurations of connectors 300 and/or engagements thereof with an implantable medical device 100, 200 to form an implantable medical device system 1000 in accordance with various principles of the present disclosure, FIG. 3 illustrating only one example of an embodiment. For instance, a connector 300 may be formed integrally (as part of and an extension of) one or both of the implantable medical devices 100, 200. In some embodiments, one or more of the retention members 120, 130, 220, 230 includes an extension, such as an axial extension, referenced herein as a lip. For instance, one or both of the proximal retention members 120, 220 of the implantable medical devices 100, 200, respectively, may include a lip 128, 228, such as illustrated in FIG. 1. In some embodiments, a connector 300 is engaged with one or both of the implantable medical devices 100, 200 via a lip 128, 228 thereof. Optionally, at least the distal retention member 130 of the first implantable medical device 100 may also include a lip 138 (illustrated in FIG. 3) with which the connector 300 may be engaged. In some embodiments, a filament forming an implantable medical device 100, 200 and a lip 128, 228 thereof may extend beyond the general structure of the implantable medical device 100, 200 to form an elongated connector 300 in accordance with various principles of the present disclosure. The implantable medical devices 100, 200 may be formed separately, with a connector 300 integrally formed with one of the implantable medical devices 100, 200 and extended and coupled to the other of the implantable medical devices 100, 200 to form an implantable medical device system 1000 in accordance with various principles of the present disclosure. The connector 300 may be an extension of a filament forming one or both of the implantable medical device 100, 200. Additionally or alternatively, the connector 300 may be a separate elongated element incorporated into a portion of the implantable medical device 100, 200 (e.g., a portion of a wall thereof) and coupled thereto, such as by welding, bonding, crimping, etc. For instance, the connector may be coupled to an implantable medical device 100, 200 during application of a coating to or formation of a liner in the implantable medical device 100, 200.

An implantable medical device system 1000 formed in accordance with various principles of the present disclosure may be deployed in any of a variety of manners. For instance, the manner of deploying an implantable medical device system 1000 formed in accordance with various principles of the present disclosure may be influenced by the common anatomical region at which the implantable medical device system 1000 is to be deployed, as may be appreciated by one of ordinary skill in the art. In some embodiments, both implantable medical devices 100, 200 are implanted at the same time, whereas in other embodiments, the implantable medical devices 100, 200 are implanted sequentially, and optionally at different times. For instance, in the example of an embodiment of an environment illustrated in FIG. 1, the first implantable medical device 100 may be deployed across apposed tissue of a stomach S and a jejunum J to form an anastomosis A therebetween. In order to allow tissue growth between the stomach S and the jejunum J to form a natural (tissue) anastomosis A (between the stomach S and the jejunum J, after performing a gastrojejunostomy), the second implantable medical device 200 may not be implanted at the same time the first implantable medical device 100 is implanted. In some instance, the second implantable medical device 200 may be implanted several days (e.g., 10-14 days) after the first implantable medical device 100 has been implanted, such as to assure that the anastomosis has been formed and/or the first implantable medical device 100 has not become dislodged.

The connector 300 of an implantable medical device system 1000 formed in accordance with various principles of the present disclosure may be deployed and coupled with the implantable medical devices 100, 200 in any of a variety of manners, which may be influenced by the manner in which the implantable medical devices 100, 200 are deployed. For instance, if the implantable medical devices 100, 200 are implanted at the same time, then the connector 300 may be deployed with the implantable medical devices 100, 200, either coupled with one or both of the implantable medical devices 100, 200, or deployed separately from the implantable medical devices 100, 200 and then engaged therewith to couple the implantable medical devices 100, 200 together. If the implantable medical devices 100, 200 are deployed separately, the connector 300 may be delivered separately or with either of the implantable medical devices 100, 200. If delivered separately, the connector 300 may be engaged with the implantable medical devices 100, 200 once delivered to the deployment site. Alternatively, the connector 300 may be delivered already engaged with either of the implantable medical devices 100, 200. If delivered with the first-deployed implantable medical device 100, 200, the connector 300 is arranged to be accessible upon deployment of the second-deployed implantable medical devices 100, 200. If delivered with the second-deployed implantable medical device 100, 200 (engaged thereto, or delivered separately and then engaged therewith at the deployment site), then the connector 300 may be extended from the second-deployed implantable medical device 100, 200 (e.g., after deployment of the second-deployed implantable medical device 100, 200) and engaged with the other of the implantable medical devices 100, 200 to couple together the implantable medical devices 100, 200.

In accordance with various principles of the present disclosure, each of the implantable medical devices 100, 200 may be configured for one or more medical procedures, treatments, etc. For instance, implantable medical devices may be used for one or more procedures, treatments, etc., such as creating an anastomosis; occluding a body passage; passing or facilitating passage of materials through an anatomical wall; draining fluid, maintaining patency of a body passage or anastomosis; etc. It will be appreciated that reference herein to occluding is to be understood as encompassing partially occluding as well. In contrast, the connector 300 is configured simply to connect the implantable medical devices 100, 200 and does not otherwise affect the procedure, treatment, etc., affected by the implantable medical devices 100, 200.

In the example of an embodiment illustrated in FIG. 1, the first implantable medical device 100 is in the configuration of a stent with a passage 105 extending therethrough to create a passage (anastomosis A) between a stomach S and a jejunum J of a patient (a gastrojejunostomy). As such, the first implantable medical device 100 may be considered to have a scaffold structure configured to maintain patency of an anastomosis A. In contrast, in the example of an embodiment illustrated in FIG. 1, the second implantable medical device 200 is in the configuration of a pyloric occlusion device having an occluded or partially occluded passage, or no passage therethrough. The implantable medical device 200 is positioned within the pylorus P of the patient and with respect to the anastomosis A formed by the first implantable medical device 100 to create a gastrojejunal bypass between the stomach S and the jejunum J. More particularly, the second implantable medical device 200 is configured to obstruct passage of gastric contents to the duodenum D (by occluding, partially or fully, the pylorus P), and thereby to redirect the gastric contents to the anastomosis A and into the jejunum J. The connector 300 of an implantable medical device system 1000 formed in accordance with various principles of the present disclosure has a different structural configuration than either of the implantable medical devices 100, 200 of the implantable medical device system 1000, and simply connects the implantable medical devices 100, 200. Both of the implantable medical devices 100, 200 are configured to resist migration out of the stomach S (which may generally be considered distal migration). In general, the distal retention member 130 of the first implantable medical device 100 and the connector 300 may assist in inhibiting distal migration of the second implantable medical device 200 (in this case, into the duodenum D). For instance, the inner wall 132 of the distal retention member 130 may anchor the first implantable medical device 100 with respect to the apposed tissue walls of the stomach S and the jejunum J to hold the first implantable medical device 100 in place with respect thereto. Similarly, the distal retention member 230 of the second implantable medical device 200 may assist in inhibiting distal migration of the first implantable medical device 100 (in this case, into the jejunum J). For instance, similar to the first implantable medical device 100, the inner wall 232 of the distal retention member 230 of the second implantable medical device 200 may anchor the second implantable medical device 200 in place with respect to its deployment site and thereby, via the connector 300, also anchor the first implantable medical device 100 against distal migration. Similar to the distal retention member 130 of the first implantable medical device 100, the distal retention member 230 of the second implantable medical device 200 may include an outer wall 234 generally spaced apart from the inner wall 232, with a peripheral wall 236 extending therebetween (in this case optionally contacting tissue of the duodenum D, in contrast with the peripheral wall 136 of the distal retention member 130 of the first implantable medical device 100).

In accordance with another aspect of the present disclosure, in the example of an embodiment illustrated in FIG. 1, the connection of the first implantable medical device 100 with the second implantable medical device 200 may also create a safety mechanism for the gastrojejunal bypass facilitated by the illustrated example of an embodiment of an implantable medical device system 1000. More particularly, by virtue of the connection of the implantable medical devices 100, 200 via the connector 300, if the first implantable medical device 100 migrates from its deployment site, and the anastomosis A thus closes, the first implantable medical device 100 also causes the second implantable medical device 200 to migrate (proximally) out of its deployment site in the pylorus P. As such, although the anastomosis A between the stomach S and the jejunum J may close, gastric materials would no longer be prevented from exiting the stomach S via the pylorus P, as the displaced second implantable medical device 200 no longer occludes the pylorus P.

It will be appreciated that implantable medical devices 100, 200 used in an implantable medical device system 1000 formed in accordance with various principles of the present disclosure may have other configurations, as may be readily appreciated by those of ordinary skill in the art. Various anti-migration features such as known to those of ordinary skill in the art may be provided to resist migration (e.g., anchor) the respective implantable medical devices 100, 200 with respect to the deployment sites thereof.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

Various further benefits of the various aspects, features, components, and structures of an implantable medical device system such as described above, in addition to those discussed above, may be appreciated by those of ordinary skill in the art.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for forming a gastric bypass in a patient, said system comprising:
a first implantable medical device configured to form an anastomosis between the stomach and a portion of the jejunum of the patient;
a second implantable medical device configured to occlude the pylorus of the patient; and
a connector coupling said first implantable medical device and said second implantable medical device.

2. The system of claim 1, wherein said connector has a configuration distinct from the configurations of said first implantable medical device and said second implantable medical device.

3. The system of claim 2, wherein said connector is elongated and has a length capable of extending from said first implantable medical device when implanted to create an anastomosis between the stomach and the jejunum and said second implantable medical device when implanted across the pylorus.

4. The system of claim 1, wherein:
said first implantable medical device is a stent configured to maintain patency of the anastomosis, said stent comprising a saddle region configured to extend through the stomach wall and a portion of a wall of the jejunum, a proximal retention member extending radially outwardly from a proximal end of said saddle region and configured to be seated within the stomach to inhibit distal migration of said first implantable medical device away from the stomach, and a distal retention member extending radially outwardly from a distal end of said saddle region and configured to be seated within the jejunum to inhibit proximal migration of said first implantable medical device into the stomach;
said second implantable medical device has a saddle region configured to occlude flow of material therethrough, a proximal retention member extending radially outwardly from a proximal end of said saddle region and configured to be seated within the stomach to inhibit distal migration of said second implantable medical device away from the stomach, and a distal retention member extending radially outwardly from a distal end of said saddle region and configured to be seated within the duodenum of the patient to inhibit proximal migration of said second implantable medical device into the stomach; and
said connector is an elongated element coupling said first implantable medical device and said second implantable medical device.

5. The system of claim 4, wherein said connector is coupled to a proximal retention member of at least one of said first implantable medical device and said second implantable medical device.

6. The system of claim 5, wherein said connector includes an elongated portion having a first end and a second end, and a connector bar coupled to one of the first end or the second end of said elongated portion and movable between a first position extending along said elongated portion and a second position transverse to said elongated portion, said connector bar being extendable through a portion of a wall of one of said first implantable medical device or said second implantable medical device when in the first position, and movable to the second position to hold the one of the first end or the second end of said elongated portion coupled to the portion of a wall of one of said first implantable medical device or said second implantable medical device.

7. The system of claim 6, wherein:
said one of said first implantable medical device or said second implantable medical device has at least one double-walled retention member comprising an inner wall adjacent said saddle region and an outer wall spaced away from said inner wall and defining a space between said inner wall and said outer wall; and
said connector bar is extendable into the space between said inner wall and said outer wall of said double-walled retention member and movable to the second position to be retained in the space.

8. The system of claim 1, wherein said connector is formed separately from said first implantable medical device and said second implantable medical device.

9. The system of claim 1, wherein:
said first implantable medical device anchors, via said connector, said second implantable medical device against migrating distally from the stomach; and
said second implantable medical device anchors, via said connector, said first implantable medical device against migrating distally from the stomach.

10. The system of claim 1, wherein said first implantable medical device displaces said second implantable medical device upon migrating from the anastomosis to open the pylorus for passage of gastric materials therethrough.

11. A system for maintaining a pair of implantable devices in place with respect to respective deployment sites for said pair of implantable medical devices, said system comprising:
a first implantable medical device;
a second implantable medical device; and
a connector coupling said first implantable medical device and said second implantable medical device;
wherein:
the deployment sites are at anatomical environments tending to drive said first implantable medical device and said second implantable medical device apart from each other; and
each of said implantable medical devices holds the other of said implantable medical devices in place via said connector.

12. The system of claim 11, wherein said connector has a configuration distinct from the configurations of said first implantable medical device and said second implantable medical device.

13. The system of claim 12, wherein said connector is elongated and has a length sufficient to extend between said first implantable medical device and said second implantable medical device without exerting force on either of said first implantable medical device and said second implantable medical upon coupling said first implantable medical device and said second implantable medical.

14. The system of claim 11, wherein said connector is formed separately from said first implantable medical device and said second implantable medical device.

15. The system of claim 11, wherein said connector includes an elongated portion having a first end and a second end, and a connector bar coupled to one of the first end or the second end of said elongated portion and movable between a first position extending along said elongated portion and a second position transverse to said elongated portion, said connector bar being extendable through a portion of a wall of one of said first implantable medical device or said second implantable medical device when in the first position, and movable to the second position to hold the one of the first end or the second end of said elongated portion coupled to the portion of a wall of one of said first implantable medical device or said second implantable medical device.

16. A method of forming a gastric bypass, said method comprising:
implanting a first implantable device across an apposed portion of a stomach wall and portion of a jejunum to form an anastomosis between the stomach and the jejunum;
implanting a second implantable medical device in a pylorus to at least partially occlude the pylorus; and
coupling the first implantable medical device and the second implantable medical device with an elongated connector.

17. The method of claim 16, further comprising implanting the first implantable medical device on a first day, and implanting the second implantable medical device on a second day different from the first day.

18. The method of claim 16, wherein coupling the first implantable medical device and the second implantable medical device with an elongated connector is performed after implanting the first implantable medical device and the second implantable medical device.

19. The method of claim 16, further comprising anchoring the second implantable medical device against migrating distally from the stomach via the connector and the first implantable medical device, and anchoring the first implantable medical device against migrating distally from the stomach via the connector and the first implantable medical device.

20. The method of claim 16, further comprising coupling the connector between the first implantable medical device and the second implantable medical device so that the first implantable medical device displaces the second implantable medical device upon migrating from the anastomosis to open the pylorus for passage of gastric materials therethrough.
